(19) United States
(12) Patent Application Publication
(10) Pub. No.: US 2025/0120705 A1
(43) Pub. Date: Apr. 17, 2025

(54) STAPLE CARTRIDGE COMPRISING A COMPOSITE SLED

(71) Applicant: Cilag GmbH International, Zug (CH)

(72) Inventors: Frederick E. Shelton, IV, Hillsboro, OH (US); Kevin M. Fiebig, Cincinnati, OH (US); Nicholas Fanelli, Morrow, OH (US); Scott A. Jenkins, Mason, OH (US); Carlie J. Love, Loveland, OH (US); Seth D. Holdmeyer, Cincinnati, OH (US); Adam D. Hensel, Gahanna, OH (US); Jason L. Harris, Lebanon, OH (US); Taylor W. Aronhalt, Loveland, OH (US)

(21) Appl. No.: 18/379,810

(22) Filed: Oct. 13, 2023

(51) Int. Cl. A61B 17/072

(52) U.S. Cl. CPC A61B 17/072; A61B 2017/07278; A61B 2017/07285

(57) ABSTRACT

A stapling assembly comprising a cartridge body, staple drivers, staples, and a sled assembly is disclosed. The cartridge body comprises a deck, a longitudinal slot, and staple cavities defined in the deck. The sled assembly is configurable in a first configuration and a second configuration. The sled assembly is movable distally in the first configuration during a staple firing stroke. The sled assembly is movable proximally in the second configuration during a retraction stroke. The sled assembly comprises a first component and a second component. The first component comprises a base and at least one rail extending from the base. The rail comprises a distal-facing ramp configured to engage the staple drivers to eject the staples from the staple cavities during the staple firing stroke. The rail is comprised of metal. The second component is movable within the longitudinal slot. The first component is movable relative to the second component.

### BACKGROUND

The present invention relates to surgical instruments and, in various arrangements, to surgical stapling and cutting instruments, end effectors, and staple cartridges for use therewith that are designed to staple and cut tissue.

In various surgical instruments a sled of a staple cartridge is pushed distally by a firing driver to eject staples from the staple cartridge during a staple firing stroke. When the firing driver is retracted after the staple firing stroke, in some such instruments, the sled is left behind in a distal fired position. The sled can comprise a tissue cutting knife that remains exposed when the sled is left behind in its distal fired position which can accidentally contact patient tissue. As such, it may be desirable for a sled having a tissue cutting knife to be retracted to a proximal position where a sharp edge, or blade, of the knife is housed in a knife housing to prevent the blade from coming into contact with patient tissue, for example. However, the sled can become stuck as it is being retracted. For instance, one or more of the staple drivers of the staple cartridge can fall down behind the sled during the staple firing stroke which prevents the sled from being retracted. As such it may be desirable for a sled to be comprised of multiple components which can separate such that the knife can still be retracted even if the remainder of the sled cannot. Various multi-component sled and firing driver arrangements disclosed herein address such problems.

### SUMMARY

In accordance with the present disclosure, a stapling assembly comprising a cartridge body is disclosed. The cartridge body comprises a deck including a proximal end and a distal end, a longitudinal slot extending from the proximal end toward the distal end, and a plurality of staple cavities defined in the deck. The stapling assembly further comprises a plurality of staple drivers movably positioned in the staple cavities, a plurality of staples removably stored in the staple cavities, and a sled assembly configurable in a first configuration and a second configuration. The sled assembly is movable distally in the first configuration during a staple firing stroke. The sled assembly is movable proximally in the second configuration during a retraction stroke. The sled assembly comprises a first component and a second component. The first component comprises a base and at least one rail extending from the base. The rail comprises a distal-facing ramp. The distal-facing ramp is configured to engage the staple drivers to eject the staples from the staple cavities during the staple firing stroke. The rail is comprised of metal. The second component is movable within the longitudinal slot. The first component is movable relative to the second component.

### LISTING OF THE FIGURES

Various features of the embodiments described herein, together with advantages thereof, may be understood in accordance with the following description taken in conjunction with the accompanying drawings as follows:

FIG. 1 is a partial perspective view of a firing driver and a sled assembly of a surgical stapling instrument having a knife portion and a wedge portion in accordance with the present disclosure;

FIG. 2 is a cross-sectional elevation view of the firing driver and sled assembly of FIG. 1;

FIG. 3 is a cross-sectional plan view of the firing driver and sled assembly of FIG. 1;

FIG. 4 is a cross-sectional view of an end effector including a staple cartridge and the firing driver and sled assembly of FIG. 1 depicting the sled assembly ejecting staples from the staple cartridge during a staple firing stroke;

FIG. 5 is a cross-sectional view of the end effector of FIG. 4 depicting the sled assembly in a distal fired position after the staple firing stroke has been completed;

FIG. 6 is a cross-sectional view of the end effector of FIG. 4 depicting a wedge portion of the sled assembly disengaging from the firing driver during a retraction stroke of the firing driver;

FIG. 7 is a perspective view of a staple cartridge for use with a surgical stapling instrument in accordance with the present disclosure, wherein the staple cartridge comprises a longitudinal slot comprising recesses and protrusions;

FIG. 7A is a partial cross-sectional perspective view of the staple cartridge of FIG. 7 illustrating a sled assembly of the staple cartridge including a wedge component and a knife component.

FIG. 8 is a perspective view of the sled assembly of FIG. 7A depicting the knife component of the sled assembly disengaged from the wedge component of the sled assembly;

FIG. 9 is a perspective view of the sled assembly of FIG. 7A depicting the knife component of the sled assembly engaged with the wedge component of the sled assembly;

FIG. 10 is a perspective view of a multi-component sled for use with a staple cartridge in accordance with the present disclosure;

FIG. 11 is an exploded view of the multi-component sled of FIG. 10;

FIG. 12 is a cross-sectional view of the multi-component sled of FIG. 10;

FIG. 13 is a bottom perspective view of the multi-component sled of FIG. 10;

FIG. 13A is a perspective view of a multi-material sled in accordance with the present disclosure;

FIG. 14 is a partial perspective view of a staple cartridge comprising a sled in accordance with the present disclosure depicting the sled in a distal fired position;

FIG. 15 is a partial cross-sectional view of an end effector of a surgical instrument including the staple cartridge of FIG. 14;

FIG. 16 is a perspective view of the sled of the staple cartridge of FIG. 14;

FIG. 17 is another perspective view of the sled of the staple cartridge of FIG. 14;

FIG. 18 is a cross-sectional view of the sled of the staple cartridge of FIG. 14 depicting a knife of the sled in an exposed configuration;

FIG. 19 is a cross-sectional view of the sled of the staple cartridge of FIG. 13 depicting the knife of the sled in a retracted configuration;

FIG. 20 is a cross-sectional view of an end effector comprising a sled of a staple cartridge including a knife housing in accordance with the present disclosure;

FIG. 21 is a cross-sectional view of the end effector of FIG. 20 depicting a knife of the sled positioned at least partially within the knife housing;

FIG. 22 is a perspective view of the staple cartridge of FIG. 20 depicting the knife of the sled of the staple cartridge positioned at least partially within the knife housing;

FIG. 23A is a partial cross-sectional view of a staple cartridge comprising a sled and a separate tissue cutting knife in accordance with the present disclosure; and

FIG. 23B is a partial cross-sectional view of the staple cartridge of FIG. 23A illustrating the tissue cutting knife in a recessed configuration at the end of a staple firing stroke.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

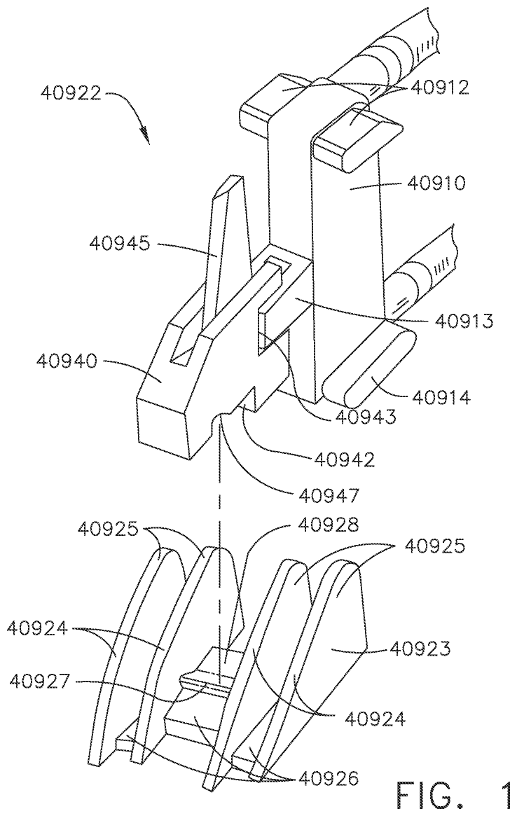

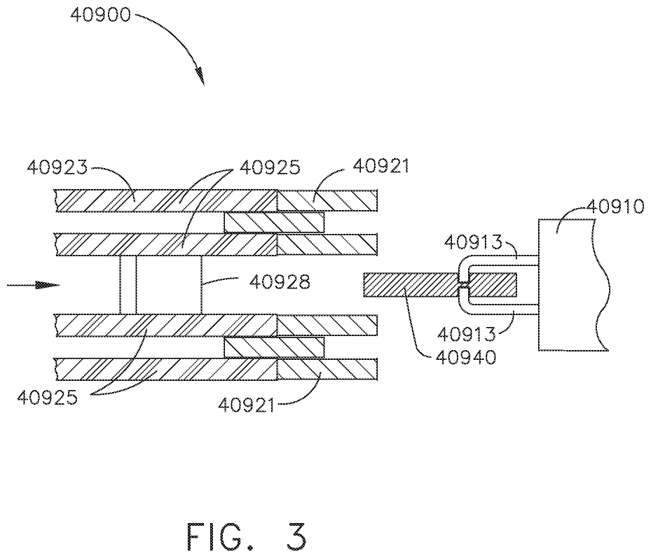

FIGS. 1-6 illustrate a surgical instrument 40900 comprising an end effector 40930 configured to receive a staple cartridge 40920 and a firing driver 40910. The staple cartridge 40920 is replaceable, i.e., the staple cartridge 40920 is readily removable from the end effector 40930 during a surgical procedure and replaceable with another staple cartridge; however, the staple cartridge 40920 may not be replaceable. The end effector 40930 comprises a first jaw 40931 and a second jaw 40932 movable relative to the first jaw 40931 to capture tissue in between the first jaw 40931 and the second jaw 40932. However, the first jaw 40931 can be movable relative to the second jaw 40932. The staple cartridge 40920 comprises a plurality of staple drivers 40921 and a sled assembly 40922 movable distally to eject staples 40929 from the staple cartridge 40920 during a firing stroke of the firing driver 40910. Referring to FIG. 3, the staple drivers 40921 are triple staple drivers configured to support three staples thereon. Further, the staple drivers 40921 are positioned on both sides of a longitudinal slot defined in the staple cartridge 40920 and are configured to eject staples 40929 from a plurality of longitudinal rows of staple cavities defined in the staple cartridge 40920.

Further to the above, the firing driver 40910 comprises a first lateral cam 40914 configured to engage the first jaw 40931 and a second lateral cam 40912 configured to engage the second jaw 40932. The first jaw 40931 comprises a longitudinal cavity 40933 defined therein that is configured to receive the first cam 40914 of the firing driver 40910. Further, the second jaw 40932 comprises a longitudinal cavity 40934 defined therein that is configured to receive the second cam 40912 of the firing driver 40610. During the firing stroke, the first cam 40914 and the second cam 40912 are configured to maintain the position of the first jaw 40931 relative to the second jaw 40932.

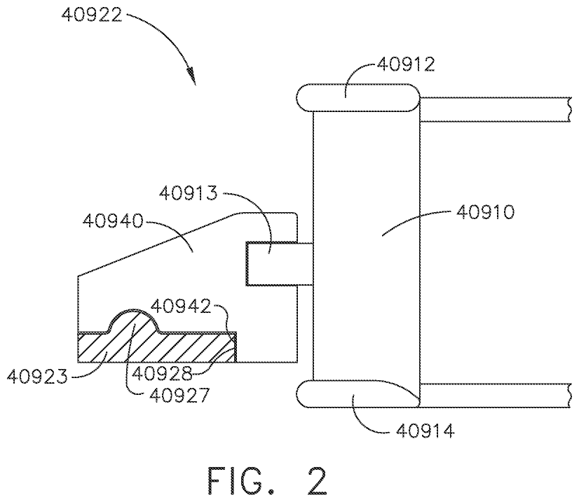

Further to the above, referring to FIGS. 1 and 2, the sled assembly 40922 comprises a wedge portion 40923 and a knife portion 40940. The wedge portion 40923 comprises a plurality of rails 40925 interconnected by a base 40926. Each of the rails 40925 comprises a distal ramp 40924 facing distally that is configured to cammingly engage the staple drivers 40921 of the staple cartridge 40920 to eject the staples 49023 from the staple cartridge 40920 during the firing stroke. The knife portion 40940 is positioned on and connected to the wedge portion 40923 as illustrated in FIG. 2. Specifically, the knife portion 40940 comprises a cutout 40947 defining a distal wall 40942 and the base portion 40926 of the wedge portion 40923 comprises a central protrusion 40927 received in the cutout 40947 of the knife portion 40940 as illustrated in FIG. 2. The cutout 40947 of the knife portion 40940 is sized and shaped to receive the complementary central protrusion 40927 of the sled 40922. The cutout 40947 of the knife portion 40940 and the central protrusion 40927 of the sled 40922 releasably interlock the knife portion 40940 and the wedge portion 40923 together, as discussed in greater detail further below.

In use, the staple cartridge 40920 is seated in the first jaw 40931 of the end effector 40930 with the sled assembly 40922 positioned in a proximal unfired position and the firing driver 40910 positioned proximal to the sled assembly 40922. The firing driver 40910 comprises opposing arms 40913 that each comprises a resilient cantilever configured to flex laterally outwardly and resiliently return laterally inwardly. The arms 40913 may be part of a metal clip, for example. At the outset of the firing stroke, the firing driver 40910 is advanced distally toward the sled assembly 40922 such that the arms 40913 engage and attach the firing driver 40910 to the knife portion 40940 of the sled assembly 40922. More specifically, each of the arms 40913 comprises a shoulder that snap-fits into a recess 40943 defined in the proximal end of the knife portion 40940 during the firing stroke of the firing driver 40910. That said, the firing driver 40910 can be configured to connect to the knife portion 40940 in any suitable manner. As the knife portion 40940 of the sled assembly 40922 is pushed distally by the firing driver 40910, the distal wall 40942 of the knife portion 40940 abuts a proximal wall 40928 of the wedge portion 40923 (see FIG. 2) such that the firing driver 40910 advances the wedge portion 40923 and the knife portion 40940 together distally. During the firing stroke, the distal ramps 40924 of the sled 40922 engage and move the staple drivers 40921 from unfired positions to fired positions to eject the staples 40929 from the staple cartridge 40920. After the staple firing stroke has been completed, or at least partially completed, the firing driver 40910 is pulled proximally to retract the firing driver 40910 and the sled assembly 40922 to their proximal unfired positions. When the now-spent staple cartridge 40920 is removed from the end effector, the sled assembly 40922 detaches from the firing driver 40910. At such point, a new, or unspent staple cartridge, can be seated in the end effector.

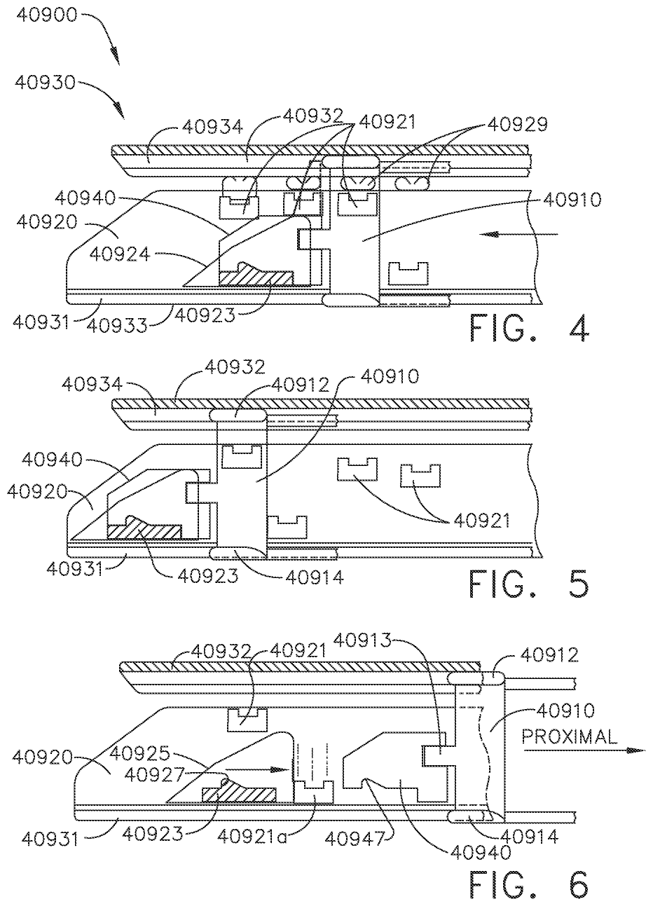

Further to the above, the sled assembly 40922 may become stuck and may not be retractable after the firing stroke. For instance, one or more of the drivers 40921 may fall from their fired position toward their unfired position behind the sled assembly 40922 after the sled assembly 40922 has passed thereby during the firing stroke. FIG. 6 illustrates a staple driver 40921a of the staple drivers 40921 which is in its unfired position after the firing stroke has been completed which could block the sled assembly 40922 from being retracted. Absent more, the tissue cutting knife 40945 would be exposed if the end effector were to be re-opened at such point. To address this potential issue, the knife portion 40940 of the sled assembly 40922 can detach from the wedge portion 40923 when the sled assembly 40922 is being retracted if the wedge portion 40923 gets stuck or blocked by a staple driver 40921, such as the staple driver 40921a, for example. When the firing driver 40910 is retracted with enough force, the engagement between the rails 40925 and the driver 40921a causes the central protrusion 40927 of the wedge portion 40923 to move out of the cutout 40947 of the knife portion 40940 to separate the wedge portion 40923 and the knife portion 40940. The knife portion 40940 is then retracted proximally by the firing driver 40910—leaving behind the wedge portion 40923—to complete the retraction stroke. As such, in the event one or more staple drivers 40921 are in the path of the rails 40925 of the sled assembly 40922 when the sled assembly 40922 is retracted, the knife 40945 and the firing driver 40910 can still be retracted to their proximal unfired positions.

Further to the above, the wedge portion 40923 may comprise a first material and the knife portion 40940 comprises a second material that is different than the first material. For example, the wedge portion 40923 is comprised of plastic and the knife portion 40940 is comprised of metal. The wedge portion 40923 may be comprised of polyvinylchloride, for example, and the blade of the knife portion 40940 may be comprised of stainless steel, for example. The knife portion 40940 may comprise a plastic material and the wedge portion 40923 may comprise a metal material.

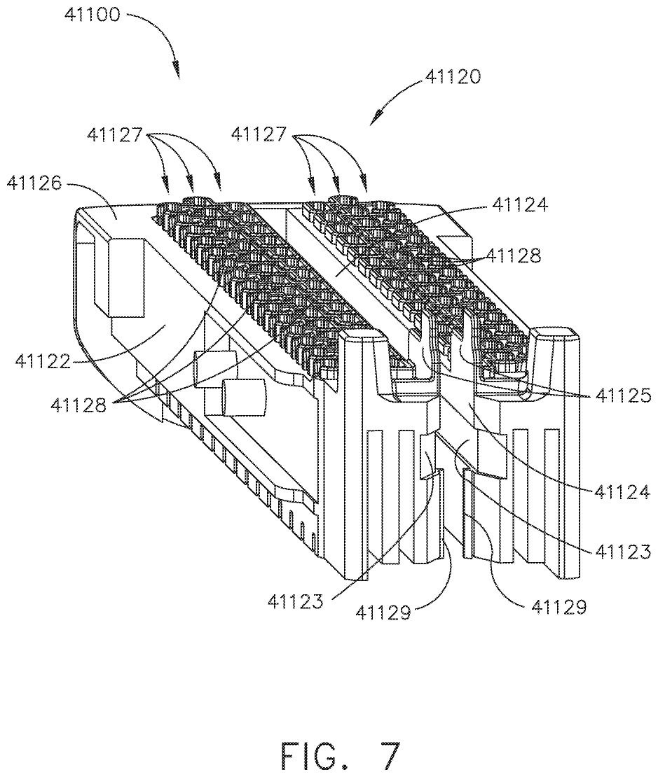

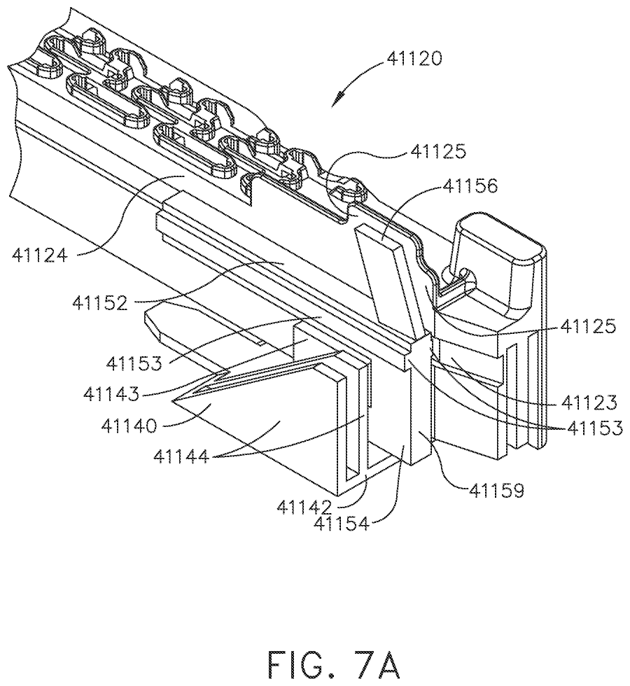

FIGS. 7-9 illustrate a stapling system 41100 comprising an end effector configured to receive a staple cartridge 41120. The staple cartridge 41120 is replaceable; however, the staple cartridge 41120 may not be replaceable. The staple cartridge 41120 comprises a cartridge body 41122 and a sled assembly 41130 movable within the cartridge body 41122. The cartridge body 41122 comprises a deck 41126, a longitudinal slot 41124, and a plurality of longitudinal rows of staple cavities 41127 defined in the deck 41126. Three rows of staple cavities 41127 are positioned on a first side of the longitudinal slot 41124 and three rows of staple cavities 41127 are positioned on a second side of the longitudinal slot 41124. Each staple cavity 41127 comprises at least one staple cavity extender 41128 extending from the deck 41126 that at least partially surrounds the staple cavity 41127. That said, only some of the staple cavities 41127 may have staple cavity extenders 41128, or none of the staple cavities 41127 may have staple cavity extenders. The staple cartridge 41120 further comprise a plurality of staple drivers movably positioned within the staple cavities 41127 that support staples thereon. Further, the staple cartridge 41120 comprises a longitudinal recess 41123 defined in the body portion 41122 on each side of the longitudinal slot 41124, a cartridge slot protrusion 41129 extending into the longitudinal slot 41124 on each side of the longitudinal slot 41124 at the proximal end thereof, and a knife housing portion 41125 extending upwardly from the deck 41126 at the proximal end of the longitudinal slot 41124. The longitudinal recesses 41123 and the cartridge slot protrusions 41129 are configured to interact with the sled assembly 41130, as discussed in greater detail below.

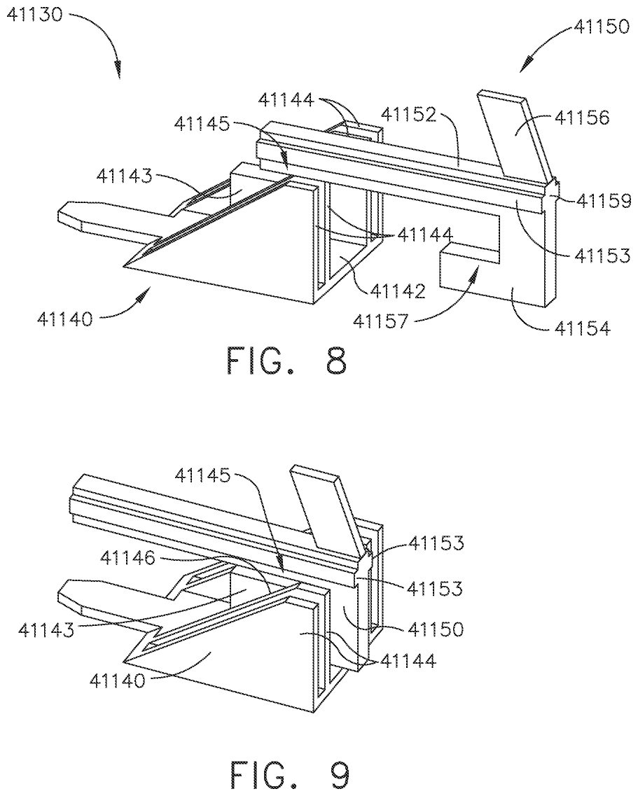

Referring to FIGS. 8 and 9, the sled assembly 41130 comprises a wedge portion 41140 and a knife portion 41150. The wedge portion 41140 comprises a base 41142, a central portion 41143, and a plurality of rails 41144 extending from the base 41142. The central portion 41143 comprises a proximal protrusion 41145 extending proximally from the central portion 41143 wherein two of the rails 41144 are positioned on each side of the central portion 41143. Each of the rails 41144 comprises a distal ramp 41146 facing distally. The distal ramps 41146 are configured to cammingly engage the staple drivers positioned in the cartridge body 41122 to eject the staples from the staple cartridge 41120. The knife portion 41150 comprises a longitudinal nose portion 41152, a hook portion 41154 extending below the nose portion 41152, and a knife, or blade, 41156 extending above the nose portion 41152. The hook portion 41154 defines an opening 41157 in the knife portion 41150 which, as discussed in greater detail below, is configured to receive a portion of the wedge portion 41140 therein.

Further to the above, a firing driver, such as the firing driver 40910 discussed above, for example, engages the knife portion 41150 of the sled assembly 41130 during a firing stroke to advance the knife portion 41150 and the wedge portion 41140 together from a proximal unfired position to a distal fired position. As the knife portion 41150 is pushed distally by the firing driver, the knife portion 41150 pushes the wedge portion 41140 distally owing to the engagement of the hook portion 41154 of the knife portion 41150 with the proximal protrusion 41145 of the wedge portion 41140. After the firing stroke has been completed, or at least partially completed, the firing driver can be retracted so that the jaws of the stapling system 41100 can be re-opened and/or so that the stapling system 41100 can be reset. As such, the firing driver retracts the knife portion 41150 proximally to its original proximal unfired position within the staple cartridge 41120. The firing driver and/or the knife portion 41150 comprise one or more features that couple the firing driver and knife portion 41150 so that the firing driver can retract the knife portion 41150. The wedge portion 41140, however, remains in the distal fired position within the staple cartridge 41120 and is not retracted with the knife portion 41150. The interface between the knife portion 41150 and the wedge portion 41140 permits the knife portion 41150 to slide proximally away from the wedge portion 41140 as the knife portion 41150 is being retracted.

Further to the above, the knife portion 41150 can rest upon and/or can be supported by the wedge portion 41140 during the staple firing stroke. When the knife portion 41150 is retracted proximally relative to the wedge portion 41140 during the retraction stroke, however, the knife portion 41150 can no longer be supported by the wedge portion 41140. That being said, the cartridge body 41122 is configured to support the knife portion 41150 during the staple firing stroke and the retraction stroke. More specifically, the knife portion 41150 comprises lateral protrusions 41153 that are received within and are supported by the sidewalls of longitudinal recesses 41123 defined in the cartridge body 41122 during the staple firing and retraction strokes. The lateral protrusions 41153 extend longitudinally along the lateral sides of the nose portion 41152 and slide within the longitudinal recesses 41123. Owing to the configuration of the lateral protrusions 41153 and the longitudinal recesses 41123, the knife portion 41150 is prevented from, or at least limited in, moving vertically within the longitudinal slot 41124 of the cartridge body 41122. In any event, the knife, or blade, 41156 of the knife portion 41150 is housed within the knife housing portion 41125 of the cartridge body 41122 when the knife portion 41150 is in its proximal unfired position.

When the knife portion 41150 is retracted into its proximal unfired position by the firing driver, a proximal end 41159 of the knife portion 41150 engages the cartridge slot protrusions 41129 of the cartridge body 41122 which stops the proximal movement of the knife portion 41150. The firing driver is then retracted further proximally into its unfired position which causes the firing driver to detach from the knife portion 41150. As such, the sled assembly 41130 of the staple cartridge 41120 is not attached to the firing driver of the stapling system 41100 when the staple cartridge 41120 is removed from the stapling system 41100. Moreover, the knife, or blade, 41156 of the knife portion 41150 is stored or stowed within the knife housing 41125 of the cartridge body 41122 so that the tissue cutting edge of the knife 41156 is not accidentally touched by a clinician handling the staple cartridge 41120.

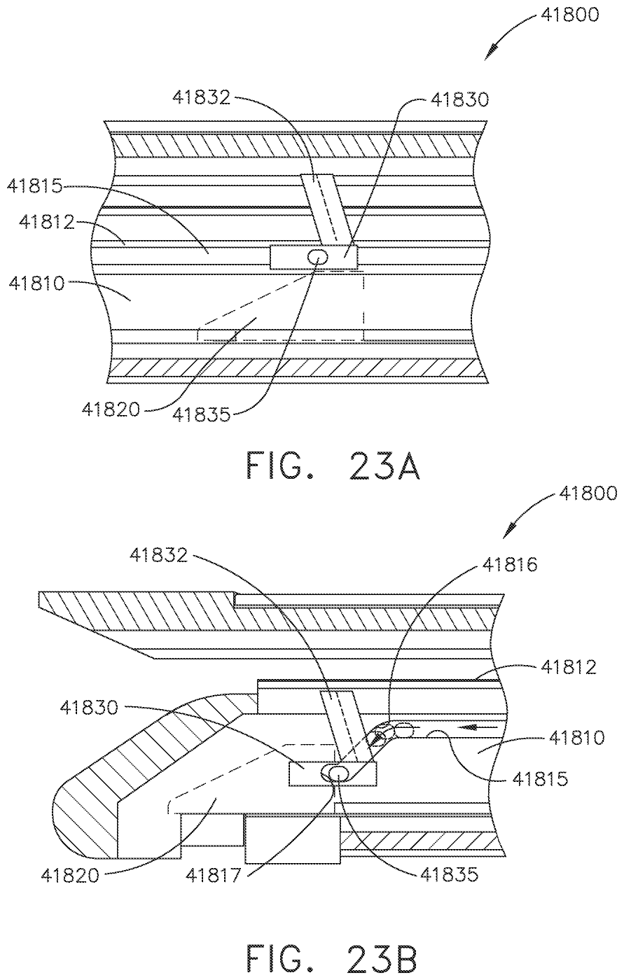

A staple cartridge 41800 is depicted in FIGS. 23A and 23B that is usable with a surgical stapling instrument comprising a firing driver. The staple cartridge 41800 comprises a cartridge body 41810 including a deck 41812 configured to support patient tissue and staple cavities defined in the deck 41812, staple drivers movable within the staple cavities, and staples removably stored in the staple cavities that are driven by the staple drivers during a staple firing stroke. The staple cartridge 41800 further comprises a sled 41820 moveable from a proximal unfired position to a distal fired position (FIG. 23B) by the firing driver during the staple firing stroke. Similar to the other sleds disclosed herein, the sled 41820 engages the staple drivers during the staple firing stroke to eject the staples from the staple cartridge 41800 and deform the staples against an anvil positioned opposite the staple cartridge 41800. The staple cartridge 41800 further comprises a tissue cutting knife 41830 that is also pushed distally by the firing driver during the staple firing stroke. Notably, the tissue cutting knife 41830 is not connected to the sled 41820 but it does move with the sled 41820 during the staple firing stroke. During the staple firing stroke, referring to FIG. 23A, a knife blade 41832 of the tissue cutting knife 41830 extends above the deck 41812 of the cartridge body 41810 such that the knife blade 41832 incises patient tissue positioned intermediate the anvil and the deck 41812 of the staple cartridge 41800. At the end of the staple firing stroke, referring to FIG. 23B, the tissue cutting knife 41830 descends, or dives, downwardly within the cartridge body 41810 such that the knife blade 41832 is positioned below the top surface of the deck 41812. Such downward motion of the tissue cutting knife 41830 is controlled by one or more cams defined on the cartridge body. More specifically, the cartridge body 41810 comprises a cam slot 41815 defined therein and the tissue cutting knife 41830 comprises a cam pin 41835 extending into the cam slot 41815 that moves within the cam slot 41815 during the staple firing stroke and is moved downwardly by a sidewall 41816 of the cam slot 41815 when the tissue cutting knife 41830 reaches the end of the staple firing stroke. As illustrated in FIG. 23B, the sidewall 41816 of the cam slot 41815 guides the cam pin 41835 into a dwell 41817 that prevents the blade 41832 of the tissue cutting knife 41830 from extending above the deck 41812. The cartridge body 41810 comprises at least one bump or detent that holds the cam pin 41835 in the dwell 41817 so that the tissue cutting knife 41830 cannot move back up into its raised position. Once the cam pin 41835 is in the dwell 41817 and, accordingly, the blade 41832 is recessed below the deck 41812, the firing driver of the surgical stapling instrument can be retracted leaving behind the sled 41820 in its distal fired position and the tissue cutting knife 41830 in its descended position. As a result, the knife blade 41832 is not exposed and is unlikely to accidentally come into contact with the patient tissue and/or the clinician when the clinician is removing the staple cartridge from the patient and/or removing the staple cartridge from the surgical stapling instrument.

Further to the above, the wedge portion 41140 may comprise a first material and the knife portion 41150 may comprise a second material that is different than the first material. For example, the wedge portion 41140 may comprise a plastic material and the knife portion 41150 may comprise a metal material. However, the knife portion 41150 may comprise a plastic material and the wedge portion 41140 may comprise a metal material.

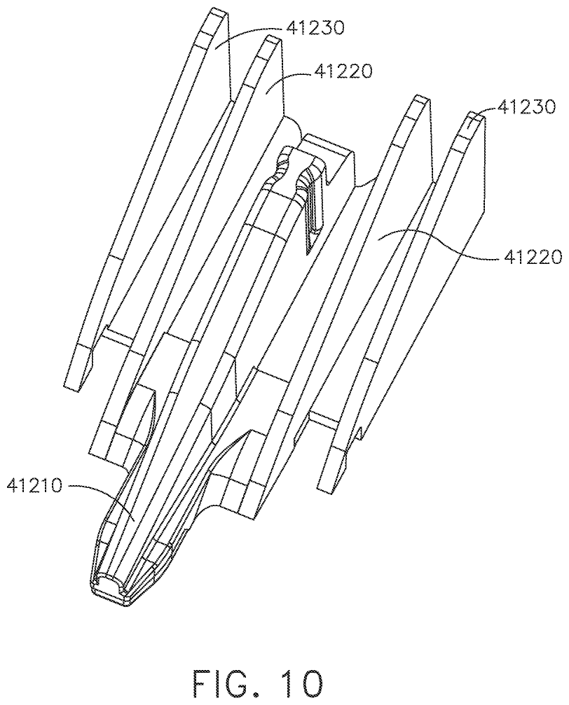

FIGS. 10-13 illustrate a multi-component sled assembly 41200 for use with a staple cartridge such as the staple cartridges described herein, for example. The sled assembly 41200 comprises a central nose component 41210, an inner rail component 41220, and an outer rail component 41230. The central nose component 41210 comprises a proximal ledge 41212 and a distal ramp 41216 configured to move within a longitudinal slot of the staple cartridge. The proximal ledge 41212 is configured to support a distal nose portion of a firing driver to defeat a firing driver lockout, as described herein. The central nose component 41210 further comprises a bottom surface 41211, a cavity 41213 defined in the bottom surface 41211, and a post 41215. In FIG. 13, the post 41215 is unformed and comprises a cylindrical shape, for example, but can comprise any suitable shape. As discussed further below, the post 41215 is heat-staked during a manufacturing process to connect the central nose component 41210, the inner rail component 41220, and the outer rail component 41230.

Further to the above, the inner rail component 41220 comprises an inner base 41222 and inner rails 41224 extending upward from the inner base 41222. Each of the inner rails 41224 comprises an inner distal ramp 41226. The inner rail component 41220 further comprises a central tab 41223 extending upward from the inner base 41222 and a through hole 41227 extending through the base 41222. The outer rail component 41230 comprises an outer base 41232 and outer rails 41234 extending upward from the outer base 41232. Each of the outer rails 41234 comprises an outer distal ramp 41236. The outer base 41232 further comprises a through hole 41237 defined through the outer base 41232.

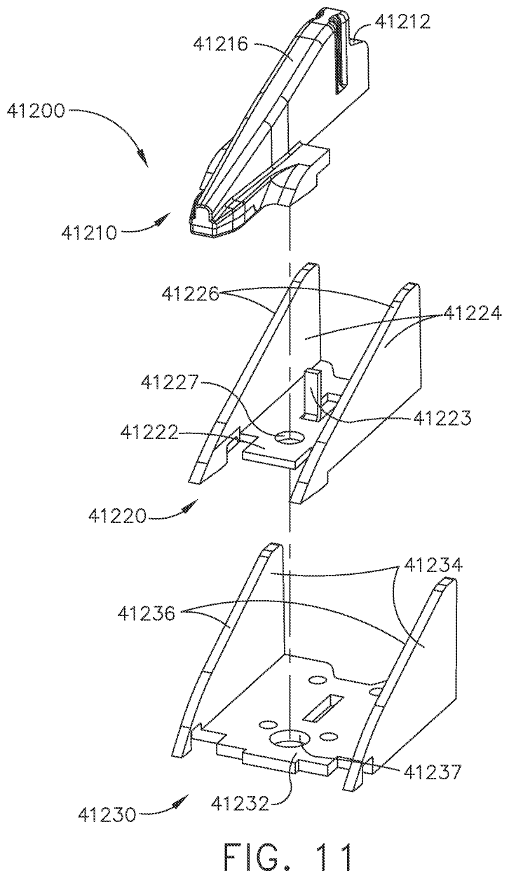

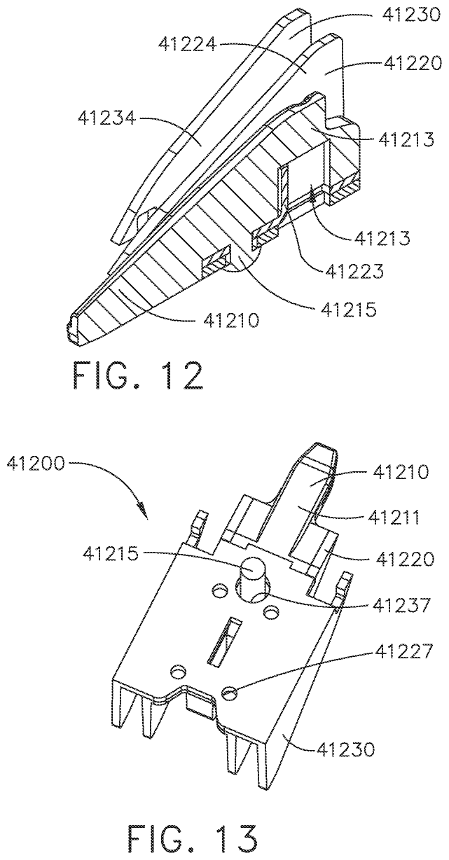

To assemble the sled assembly 41200, further to the above, the inner rail component 41220 is placed onto the outer rail component 41230 such that the through hole 41227 of the inner rail component 41220 aligns with the through hole 41237 of the outer rail component 41230. The central nose component 41210 is then placed onto the inner rail component 41220 such that the central tab 41223 of the inner rail component 41220 is received in the cavity 41213 of the central nose component 41210 to align the inner rail component 41220 with the central nose component 41210. Once assembled, the post 41215 of the central nose component 41210 extends through the through hole 41227 of the inner rail component 41220 and the through hole 41237 of the outer rail component 41230 as illustrated in FIG. 13. In FIG. 13, the post 41215 has not yet been heat-staked and extends beyond the base 41232 of the outer rail portion 41230. The central nose component 41210 is comprised of plastic, and when heat is applied to the post 41215, the post 41215 deforms such that its diameter exceeds that of the aperture 41237 in the outer rail component 41230 and the aperture 41227 in the inner rail component 41220 which locks the central nose component 41210, the inner rail component 41220, and the outer rail component 41230 to one another as illustrated in FIG. 12. The central tab 41223 of the inner rail component 41220 can be press-fit into the cavity 41213 of the central nose component 41210 to aid in coupling the central nose component 41210 to the inner rail component 41220.

In use, as discussed above, the sled assembly 41200 is configured to be advanced through a staple cartridge during a staple firing stroke to cammingly engage and move the staple drivers of the staple cartridge from an unfired position to a fired position to eject staples from the staple cartridge. Specifically, the inner distal ramps 41226 and the outer distal ramps 41236 of the sled assembly 41200 are configured to cammingly engage cam drive surfaces on the staple drivers to lift the staple drivers from their unfired positions to their fired positions as the sled assembly 41200 is advanced distally from a proximal unfired position to a distal fired position by a firing driver. When the firing driver is retracted after the staple firing stroke, the sled assembly 41200 remains in a distal fired position. In other words, the firing driver is configured to advance the sled assembly 41200 but not retract the sled assembly 41200. However, a firing driver, such as the firing driver 40910, for example, can be attachable to the sled assembly 41200 to advance the sled assembly 41200 from its proximal unfired position to a distal fired position and retract the sled assembly 41200 from a distal fired position to its proximal unfired position.

Further to the above, the inner rail component 41220 and the outer rail component 41230 of the sled assembly 41200 are made from stamped metal and the central nose portion 41210 of the sled assembly 41200 is made of plastic. As discussed above, the inner rail component 41220 and the outer rail component 41230 contact and drive the staple drivers of a staple cartridge to eject the staples out of the staple cartridge and drive the staples against an anvil positioned opposite the staple cartridge. Large forces are transmitted through the rail components 41220, 41230 and, as such, constructing the rail components 41220, 41230 can allow the rail components 41220, 41230 to withstand the large forces without deflecting, or substantially deflecting, as a result thereof. If the rails 41224, 41234, respectively, of the rail components 41220, 41230 were to deflect significantly while driving staples against the anvil, however, the staples being driven against the anvil may not be deformed to the proper formed height. More specifically, the formed height of the deformed staples may be too tall and the deformed staples may not properly clinch the patient tissue therein. That said, the metal rail components 41220, 41230 can be stiff and the interfacing surfaces between the cartridge body and the metal rail components 41220, 41230 can resist the movement of the sled assembly 41200 relative to the cartridge body. With this in mind, the central nose component 41210 of the sled assembly 41200 is comprised of plastic and is configured to slide within the cartridge body of the staple cartridge which is also comprised of plastic. The interfacing plastic surfaces between the central nose component 41210 and the cartridge body may provide little resistance to the movement of the sled assembly 41200 relative to the cartridge body.

As described above, the central nose component 41210 is comprised of plastic. In accordance with the present disclosure, the central nose component 41210 may be over-molded onto the inner rail component 41220 and/or the outer rail component 41230 to form the multi-component sled 41200 into a one-piece, unitary structure. Furthermore, the central nose component 41210 may be heat staked onto the inner rail component 41220 and the outer rail component 41230 to form the multi-component sled 41200 into a one-piece, unitary structure. Furthermore, the inner rails 41224 of the inner rail component 41220 may comprise stamped metal and the remainder of the inner rail component may be comprised of plastic which has been over-molded and/or heat staked onto the inner rails 41224. Furthermore, the outer rails 41234 of the outer rail component 41230 may comprise stamped metal and the remainder of the outer rail component may be comprised of plastic which has been over-molded and/or heat staked onto the outer rails 41234.

FIG. 13A illustrates another example of a sled 41270 for use with a staple cartridge, such as the staple cartridges described herein, for example. The sled 41270 comprises a central nose portion 41272, a pair of outer rails 41275, and a pair of inner rails 41278. The inner rails 41278 are comprised of metal while the central portion 41272 and the outer rails 41275 are comprised of plastic. The inner rails 41278 are comprised of stamped stainless steel and the plastic comprising the central nose portion 41272 and the outer rails 41275 has been overmolded onto the inner rails 41278. The outer rails 41275 are not as stiff as the inner rails 41278 and, thus, staples formed by staple drivers driven by the outer rails 41275 may be formed to a larger formed staple height as compared to staples formed by staple drivers driven by the stiffer metal inner rails 41278. A staple cartridge can comprise two inner longitudinal row of staple drivers that are driven by the metal inner rails 41278 and two outer longitudinal rows of staple drivers—that are unconnected to the inner longitudinal rows of staple drivers—that are driven by the plastic outer rails 41275. Such a configuration may experience less resistance during the staple firing stroke than the configuration depicted in FIG. 13. Moreover, such a configuration may provide smaller formed staples that tightly clinch the patient tissue adjacent the incision in the patient tissue and larger formed staples that clinch the patient tissue further away from the incision but in a manner that sufficiently permits blood, for example, to flow to the stapled tissue. Each staple driver in the inner longitudinal rows of staple drivers may be configured to drive two staples while each staple driver in outer longitudinal rows of staple drivers may be configured to drive one staple. The metal sled rails 41278 may drive twice as many staples as the plastic sled rails 41275 during a staple firing stroke and, thus, may experience about twice the firing force therein. Given the metal material that comprises the inner sled rails 41278, the metal sled rails 41278 can endure the higher firing loads without deflecting unacceptably.

In accordance with the present disclosure, a sled rail can be comprised of metal or plastic. A sled rail can be comprised of both metal and plastic. Referring again to FIG. 13A, the inner sled rails 41278 of the sled 41270, for example, can be comprised of a metal frame and a plastic material overmolded on a portion of the metal frame. The bottom 41276 of each inner sled rail 41278 may be overmolded with plastic and the top 41377 of each inner sled rail 41278 may not overmolded with plastic. The plastic bottoms 41276 of the inner sled rails 41278 can provided a smooth surface that initially engages the staple drivers as the sled 41270 is moved distally. As the sled 41270 continues to move distally, the staple drivers move up the inner sled rails 41278 onto the metal surfaces of the inner sled rails 41278. Once the staple drivers reach the tops 41277 of the inner sled rails 41278, the staple drivers have driven the staples to their fully-formed deformed heights. In this position, the inner sled rails 41278 comprise metal columns extending under and supporting the staple drivers. The metal frames of the inner sled rails 41278 may comprise notches defined therein which receive some of the overmolded plastic and interlock the plastic and metal components.

Referring again to FIG. 13A, the top surfaces of the outer sled rails 41275 and the inner sled rails 41278 may comprise flat surfaces. As also illustrated in FIG. 13A, each sled rail 41275, 41278 comprises a constant width along the longitudinal length thereof. One or more of the sled rails 41275, 41278 may comprise chamfers and/or fillets on the lateral edges 41279 of the top surfaces which decrease the lateral width of the top surfaces. Such an arrangement can control the profile of the surfaces in contact with the staple drivers.

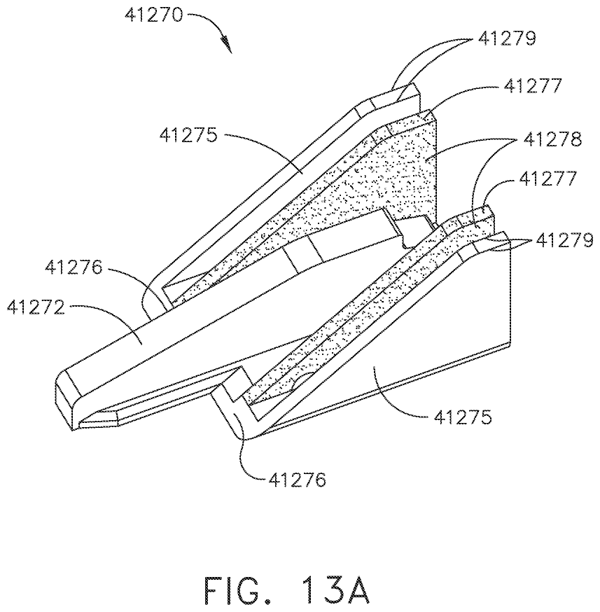

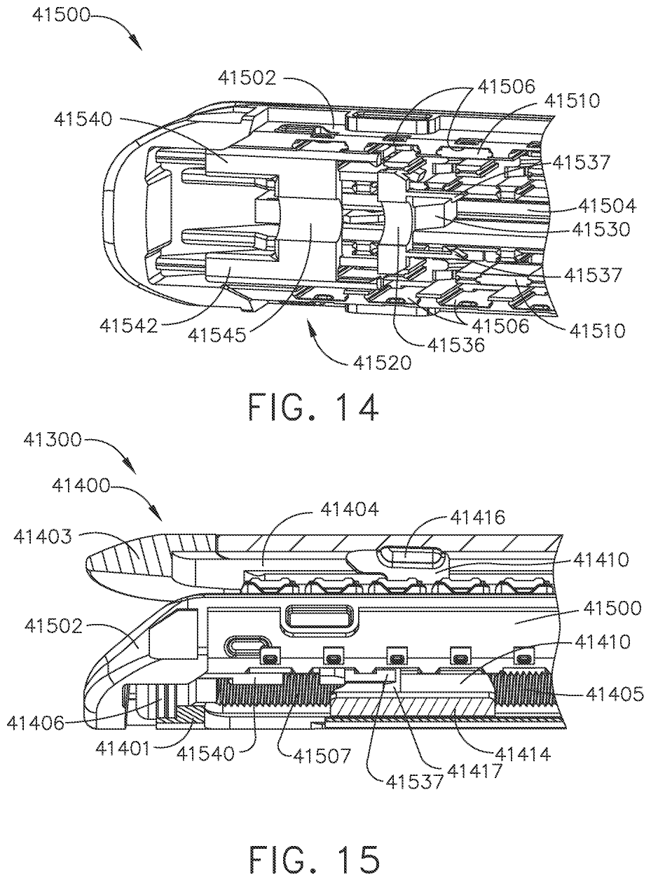

FIGS. 14-19 illustrate a surgical system 41300 comprising an end effector 41400 configured to receive a staple cartridge 41500. The end effector 41400 comprises a first jaw 41401 and a second jaw 41403 movable relative to the first jaw 41401 to capture tissue in between the first jaw 41401 and the second jaw 41403. However, the first jaw 41401 may be movable relative to the second jaw 41403. The end effector 41400 further comprises a firing driver 41410 movable relative to the first jaw 41401 and the second jaw 41403. The first jaw 41401 comprises a longitudinal drive screw 41405 rotatably supported at its distal end by a bearing 41406. The firing driver 41410 comprises a firing nut 41412, a first lateral cam 41414, a second lateral cam 41416, and lateral recesses 41417 defined on the lateral sides of the firing driver 41410. The firing nut 41412 comprises an internal thread that is threadingly engaged with external threads 41507 of the drive screw 41405 of the first jaw 41401. When the drive screw 41405 is rotated in a first direction, the firing driver 41410 is pushed distally by the drive screw 41405 and, correspondingly, the drive screw 41405 pulls the firing driver 41410 proximally when the drive screw 41405 is rotated in a second, or opposite direction. The first lateral cam 41414 is configured to cammingly engage the staple cartridge 41500 and/or the first jaw 41401 when the staple cartridge 41500 is positioned in the first jaw 41401 and the firing driver 41410 is moved from a proximal position to a distal position during a firing stroke. The second lateral cam 41416 is configured to move within a longitudinal cavity 41404 of the second jaw 41403 and cammingly engage the second jaw 41403 during the firing stroke. As such, the first and second lateral cams 41414, 41416 can approximate, or position, the first jaw 41401 relative to the second jaw 41403 during the firing stroke of the firing driver 41410.

Further to the above, the staple cartridge 41500 comprises a cartridge body 41502 including a longitudinal slot 41504 and a plurality of staple cavities 41506 defined therein. The staple cavities 41506 are arranged in a plurality of longitudinal rows in the cartridge body 41502. Specifically, three rows of staple cavities 41506 are arranged on a first side of the longitudinal slot 41504 and three rows of staple cavities 41506 are arranged on a second side of the longitudinal slot 41504 opposite the first side; however, any suitable arrangement can be utilized. The staple cartridge 41500 further comprises a plurality of staple drivers 41510 movably positioned in the cartridge body 41502. Each of the staple drivers 41510 comprises a staple support portion that moves within a staple cavity 41506 and is configured to support a staple thereon.

Further to the above, the staple cartridge 41500 comprises a multi-component sled 41520 including a nose component 41530 and a wedge component 41540 that are movable relative to one another. The wedge component 41540 comprises a base 41542 and a plurality of rails 41544 extending upward from the base 41542. Specifically, two rails 41544 are positioned on a first side of a central portion 41543 of the base 41542 and two rails 41544 are positioned on a second side of the central portion 41543 opposite the first side. The central portion 41543 of the base 41542 comprises a concave surface 41545 configured to receive a portion of the drive screw 41405—but is not threadably engaged with the drive screw 41405—when the staple cartridge 41500 is positioned in the end effector 41400. Further, each of the rails 41544 comprises a distal ramp 41546 facing distally and configured to cammingly engage the staple drivers 41510 to eject the staples from the staple cartridge 41500. The central portion 41543 comprises an upstanding body 41547 and a knife 41549 pivotably connected to the upstanding body 41547 by a pin 41548. Further, the wedge component 41540 comprises flexible tabs 41541 extending proximally from the inner most rails of the rails 41544, i.e., closest to the central portion 41543. The flexible tabs 41541 are bent inwardly toward each other.

Further to the above, the nose component 41530 of the multi-component sled 41520 comprises a base 41532 and an upstanding body 41534 extending upward from the base 41532. The base 41532 comprises a concave surface 41536 and proximally extending tabs 41537 on either side of the concave surface 41536. Each of the tabs 41537 comprises an inward detent 41537a extending toward one another. The tabs 41537 are configured to latchingly engage with the lateral recesses 41417 of the firing driver 41410, as discussed further below. Further, the concave surface 41536 is configured to receive a portion of the drive screw 41405—but is not threadably engaged with the drive screw 41405—when the staple cartridge 41500 is positioned in the end effector 41400. As such, the wedge component 41540 and the nose component 41530 of the sled 41520 are configured to be seated onto and/or over the drive screw 41405 when the staple cartridge 41500 is positioned in the end effector 41400. After the staple cartridge 41500 is seated in the end effector 41400, the firing driver 41410 can be advanced distally toward the sled 41520 to perform the staple firing stroke. As the firing driver 41410 approaches the sled 41520, the firing driver 41410 abuts proximal surfaces 41531a, 41531b of the nose component 41530 to advance the nose component 41530 distally relative the wedge component 41540.

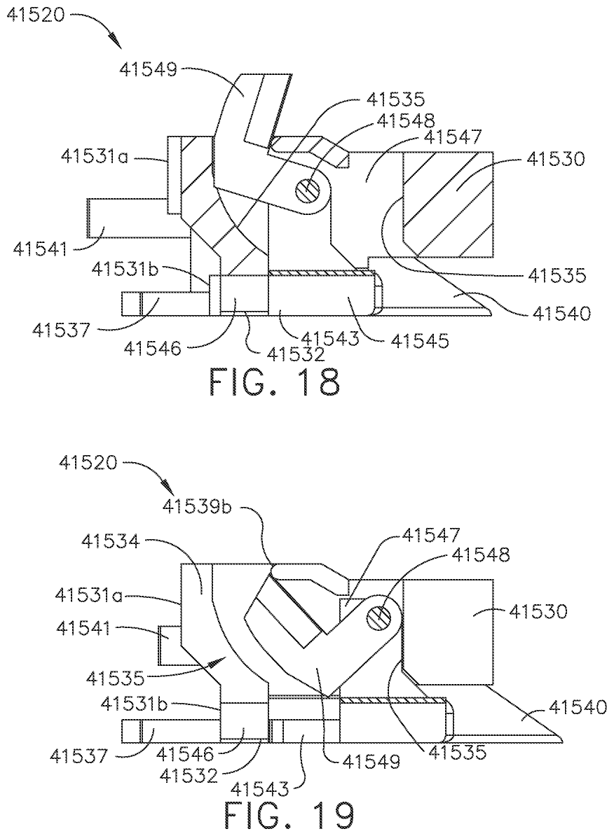

Further to the above, the upstanding body 41534 of the nose component 41530 comprises a cavity 41535 defined therein. The cavity 41535 is configured to receive the upstanding body 41547 of the wedge component 41540 therein. At the outset of the staple firing stroke, a proximal sidewall of the cavity 41535 of the nose component 41530 cammingly engages the knife 41549 of the wedge component 41540 to rotate the knife 41549 from a retracted position (FIG. 19) to an exposed position (FIG. 18). As the firing driver 41410 is advanced further distally, the base 41532 of the nose component 41530 abuts the central portion 41543 of the wedge component 41540 (see FIG. 18) such that the firing driver 41410 advances the nose component 41530 and the wedge component 41540 distally together with the knife 41549 in the exposed position. The knife 41549 of the wedge component 41540 is maintained in the exposed position by the sidewall of the cavity 41535 of the nose component 41530 during the staple firing stroke. During the staple firing stroke, further to the above, the sled 41520 engages the staple drivers 41510 to eject the staples from the staple cartridge 41500. Further, the exposed knife 41549 is configured to incise patient tissue captured in the end effector 41400 during the staple firing stroke of the firing driver 41410.

When the firing driver 41410 abuts the sled 41520 to advance the sled 41520 distally, further to the above, the proximal tabs 41537 of the nose component 41530 of the sled 41520 latchingly engage the lateral recesses 41417 of the firing driver 41410 to operably couple the nose component 41530 of the sled 41520 to the firing driver 41410. After the firing driver 41410 advances the sled 41520 distally to a completely fired, or at least partially fired position, the nose component 41530 is retracted proximally with the firing driver 41410 owing to this coupling. As the nose component 41530 is retracted proximally from the position in FIG. 18, a knife retraction surface 41539b of the nose component 41530 engages and rotates the knife 41549 of the wedge component 41540 from the exposed position (FIG. 18) to the retracted position (FIG. 19). The upstanding body 41534 of the nose component 41530 further comprises a cap 41538 comprising a slot 41539a defined therein. The slot 41539a comprises a distal knife retraction surface 41539b configured to engage and rotate the knife 41549 of the wedge component 41540 as the nose component 41530 is retracted. As discussed below, the wedge component 41540 is prevented from being retracted proximally with the nose component 41530.

As the wedge component 41540 is advanced distally during the staple firing stroke, the flexible tabs 41541 extending proximally from the wedge component 41540 are generally compressed inwardly by the cartridge body 41502. That said, the flexible tabs 41541 are afforded room to resiliently flex outwardly into the staple cavities 41506 as the flexible tabs 41541 pass by the staple cavities 41506 during the staple firing stroke. Such resilient flexing and relaxing of the flexible tabs 41541 during the staple firing stroke does not stop the staple firing stroke; however, in the event that the wedge component 41540 were to be pulled proximally after being advanced through at least a portion of the staple firing stroke, the proximal ends of the flexible tabs 41541 are configured to engage the sidewalls of a staple cavity 41506 and prevent the retraction of the wedge component 41540. As such, the nose component 41530 is retractable proximally with the firing driver 41410, but not the wedge component 41540. When the nose component 41530 is retracted back into its proximal unfired position, the nose component 41530 contacts a stop surface of the cartridge body 41502 which prevents the nose component 41530 from being further retracted proximally. At such point, however, the firing driver 41410 has not yet been returned to its proximal unfired position. When the firing driver 41410 is further retracted with enough force, the proximal tabs 41537 of the nose component 41530 disengage from the lateral recesses 41417 of the firing driver 41410 to decouple the nose component 41530 from the firing driver 41410. The firing driver 41410 can then be retracted back into its original proximal unfired position to permit the spent staple cartridge 41500 to be removed from the end effector 41400. Owing to the recessed position of the knife 41549 in such instances, the possibility of a user coming into contact with the knife 41549 while removing the staple cartridge 41500 from the end effector 41400 is reduced.

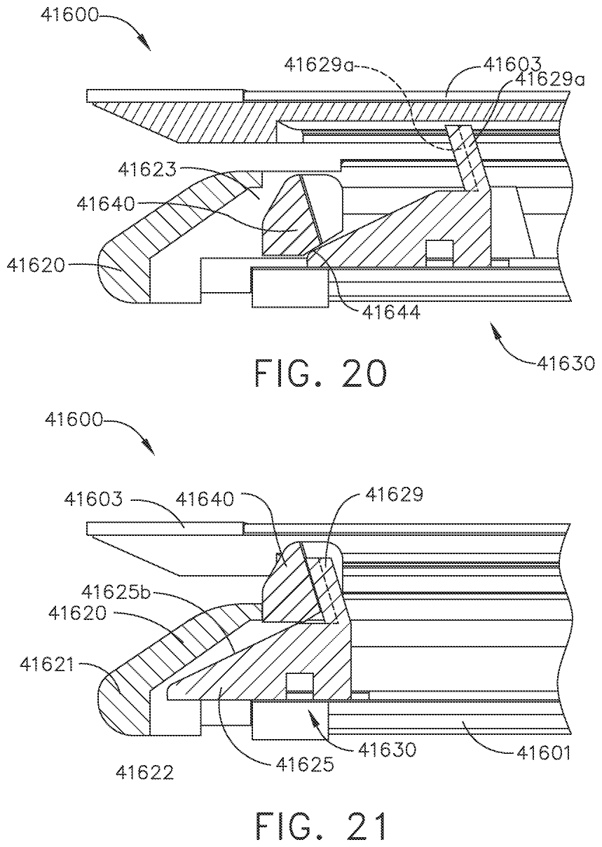

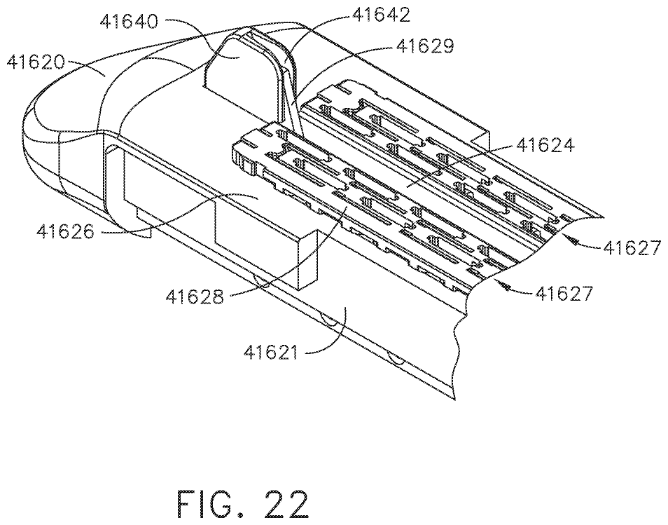

FIGS. 20-22 illustrate a surgical end effector 41600 configured to receive a staple cartridge 41620. The end effector 41600 comprises a first jaw 41601 and a second jaw 41603 movable relative to the first jaw 41601 to capture tissue in between the first jaw 40601 and the second jaw 41603. However, the first jaw 41601 may be movable relative to the second jaw 41603. The staple cartridge 41620 comprises a cartridge body 41621 and a sled 41630 movable relative to the cartridge body 41621. The cartridge body 41621 comprises a longitudinal slot 41624, a deck 41626, and a plurality of longitudinal rows of staple cavities 41627 defined in the deck 41626. Three rows of staple cavities 41627 are positioned on a first side of the longitudinal slot 41624 and three rows of staple cavities 41627 are positioned on a second side of the longitudinal slot 41624 opposite the first side. Other arrangements of staple cavities and staples are contemplated. The staple cartridge 41620 further comprise a plurality of staple drivers movably positioned within the staple cavities 41627. The staple drivers are configured to support staples thereon and are similar to the staple drivers 41510 (see FIG. 14), for example.

Further to the above, the sled 41630 of the staple cartridge 41620 is configured to be moved from a proximal unfired position to a distal fired position by a firing driver, such as the firing driver 40910 (FIG. 3), for example, during a firing stroke. The sled 41630 comprises a central nose portion 41625 and a plurality of upstanding rails, such as those described herein, on either side of the central nose portion 41625. Each of the rails comprises a distal ramp configured to cammingly engage the staple drivers of the staple cartridge 41620 to eject the staples from the cartridge body 41621. The central nose portion 41625 comprises a distal ramp 41625b and a knife 41629 extending upwardly from the central nose portion 41622. The central nose portion 41622 moves within the longitudinal slot 41624 of the cartridge body 41621 during the staple firing stroke such that the knife 41629 extends above the cartridge body 41621 to incise patient tissue captured between the first and second jaws 41601, 41603 of the end effector 41600.

Further to the above, the staple cartridge 41620 comprises a movable knife housing 41640 positioned at a distal end 41623 of the cartridge body 41621 within the longitudinal slot 41624. The knife housing 41640 comprises a proximal opening 41642 and a proximal ramp 41644 and slides within a track defined in the cartridge body 41621. The track comprises a vertical slot that extends orthogonally to the longitudinal slot 41624; however, the track can extend in any suitable direction. In use, the knife housing 41640 is movable relative to the cartridge body 41621 between a recessed positon (FIG. 20) and an extended, or exposed, position (FIG. 21). When the sled 41630 is advanced from the proximal unfired position to the distal fired position (FIG. 21), the distal ramp 41625b of the central nose portion 41625 of the sled 41630 cammingly engages the proximal ramp portion 41644 of the knife housing 41640 to lift the knife housing 41640 from the recessed position (FIG. 20) to the exposed position (FIG. 21). When the sled 41630 is in the distal fired position depicted in FIG. 21, at least a portion of the knife 41629 enters the proximal opening 41642 in the knife housing 41640. Specifically, a distal cutting edge 41629a of the knife 41629 is covered by, or enclosed within, the knife housing 41640 when the sled 41630 is in the distal fired position (FIG. 21). When the firing driver is retracted to a proximal position within the end effector 41600, the sled 41630 remains in the distal fired position with the knife housing 41640 covering the distal cutting edge 41629a of the knife 41629. As such, when the spent staple cartridge 41620 is removed from the end effector 41600, the distal cutting edge 41629a of the knife 41629 is not exposed.

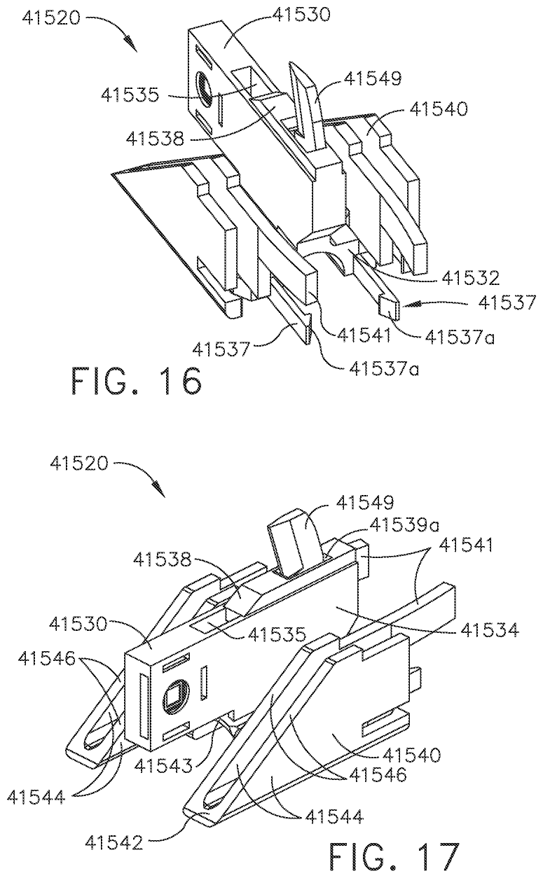

Further to the above, the sled 41630 can be prevented from moving proximally and exposing the distal cutting edge 41629a of the knife 41629 after the firing stroke is completed owing to the engagement of the sled 41630 with the cartridge body 41621. More specifically, the sled 41630 may comprise proximal engagement features, such as the flexible tabs 41541 (see FIG. 17), which prevent the sled 41630 from moving, or at least substantially moving, proximally. As such, when the staple cartridge 41620 is removed from the end effector 41600, the distal cutting edge 41629a of the knife 41629 remains covered by the knife housing 41640 and, thus, the possibility of a user coming into contact with the knife 41549 is reduced.

Various aspects of the subject matter described herein are set out in the following examples.

Example 1—A stapling assembly comprising a cartridge body (40920, 41122). The cartridge body comprises a deck (41126) including a proximal end and a distal end, a longitudinal slot (41124) extending from the proximal end toward the distal end, and a plurality of staple cavities (41127) defined in the deck. The stapling assembly further comprises a plurality of staple drivers (40921) movably positioned in the staple cavities, a plurality of staples (40929) removably stored in the staple cavities, and a sled assembly (40922, 41130) configurable in a first configuration and a second configuration. The sled assembly is movable distally in the first configuration during a staple firing stroke. The sled assembly is movable proximally in the second configuration during a retraction stroke. The sled assembly comprises a first component and a second component. The first component (40923, 41140) comprises a base (40926, 41142) and at least one rail (40925, 41140) extending from the base. The rail comprises a distal-facing ramp (40924, 41146). The distal-facing ramp is configured to engage the staple drivers to eject the staples from the staple cavities during the staple firing stroke. The rail is comprised of metal. The second component (40940, 41150) is movable within the longitudinal slot. The first component is movable relative to the second component.

Example 2—The stapling assembly of Example 1, wherein the second component is comprised of plastic.

Example 3—The stapling assembly of Examples 1 or 2, wherein the first component and the second component are coupled in the first configuration, and wherein the first component and the second component are decoupled in the second configuration.

Example 4—The stapling assembly of Example 3, further comprising a first jaw (40931), a second jaw (40932) rotatable relative to the first jaw between an unclamped position and a clamped position, and a firing driver (40910) movable distally during the staple firing stroke and movable proximally during the retraction stroke. The firing driver moves the sled assembly distally during the staple firing stroke.

Example 5—The stapling assembly of Example 4, wherein the firing driver is configured to retract the first component during the retraction stroke.

Example 6—The stapling assembly of Example 4, wherein the firing driver is configured to retract the second component during the retraction stroke.

Example 7—The stapling assembly of Example 6, wherein the firing driver is configured to operably attach to the second component during the staple firing stroke, and wherein the firing driver is configured to operably detach from the second component during the retraction stroke.

Example 8—The stapling assembly of Example 7, wherein the firing driver comprises at least one lock arm (40913) configured to releasably couple the firing driver to the second component.

Example 9—The stapling assembly of Example 6, wherein the first component is unconnected to the firing driver, and wherein the first component is not retracted with the firing driver during the retraction stroke.

Example 10—The stapling assembly of Examples 3, 4, 5, 6, 7, 8, or 9, wherein the first component comprises a protrusion (40927), wherein the second component comprises a cutout (40947) engaged with the protrusion to couple the second component with the first component when the sled assembly is in the first configuration, and wherein the cutout is disengaged from the protrusion when the sled assembly is in the second configuration.

Example 11—The stapling assembly of Example 10, wherein the rail of the first component comprises a first rail, wherein the first component further comprises a second rail extending from the base, wherein the second rail comprises a second distal-facing ramp, wherein the second distal-facing ramp is configured to engage the staple drivers to eject the staples from the staple cavities during the staple firing stroke, and wherein the protrusion extends between the first rail and the second rail.

Example 12—The stapling assembly of Examples 1, 2, 3, 4, 5, 6, 7, 8, 9, 10, or 11, wherein the second component comprises plastic overmolded onto the metal of the rail.

Example 13—The stapling assembly of Examples 1, 2, 3, 4, 5, 6, 7, 8, or 9, wherein the rail of the first component comprises a first rail, wherein the first component further comprises a second rail extending from the base, wherein the second rail comprises a second distal-facing ramp, wherein the second distal-facing ramp is configured to engage the staple drivers to eject the staples from the staple cavities during the staple firing stroke, wherein the second rail is comprised of metal, and wherein the first rail is positioned on a first lateral side of the longitudinal slot and the second rail is positioned on a second lateral side of the longitudinal slot.

Example 14—The stapling assembly of Example 13, wherein the first component further comprises a third rail extending from the base, wherein the third rail comprises a third distal-facing ramp, wherein the third distal-facing ramp is configured to engage the staple drivers to eject the staples from the staple cavities during the staple firing stroke, wherein the third rail is comprised of plastic, and wherein the third rail is positioned on a first lateral side of the longitudinal slot.

Example 15—The stapling assembly of Example 14, wherein the first rail is positioned closer to the longitudinal slot than the third rail.

Example 16—The stapling assembly of Examples 1, 2, 3, 4, 5, 6, 7, 8, 9, 10, 11, 12, 13, 14, or 15, wherein the second component comprises a knife (40945, 41156, 41549, 41629, 41832).

Example 17—The stapling assembly of Examples 1, 2, 3, 4, 5, 6, 7, 8, 9, 10, 11, 12, 13, 14, 15, or 16, wherein the staple assembly is replaceable.

Example 18—A stapling assembly comprising a cartridge body. The cartridge body comprises a deck including a proximal end and a distal end, a longitudinal slot extending from the proximal end toward the distal end, and a plurality of staple cavities defined in the deck. The stapling assembly further comprises a plurality of staple drivers movably positioned in the staple cavities, a plurality of staples removably stored in the staple cavities, and a sled assembly configurable in a first configuration and a second configuration. The sled assembly is movable distally in the first configuration during a staple firing stroke. The sled assembly is movable proximally in the second configuration during a retraction stroke. The sled assembly comprises a first component and a second component. The first component comprises a base and at least one rail extending from the base. The rail comprises a distal-facing ramp. The distal-facing ramp is configured to engage the staple drivers to eject the staples from the staple cavities during the staple firing stroke. The rail is comprised of metal. The second component is movable within the longitudinal slot. The first component is movable relative to the second component.

Example 19—The stapling assembly of Example 18, wherein the second component is comprised of plastic.

Example 20—The stapling assembly of Examples 18 or 19, wherein the first component and the second component are coupled in the first configuration, and wherein the first component and the second component are decoupled in the second configuration.

Example 21—The stapling assembly of Example 20, further comprises a first jaw, a second jaw rotatable relative to the first jaw between an unclamped position and a clamped position, and a firing driver movable distally during the staple firing stroke and movable proximally during the retraction stroke. The firing driver moves the sled assembly distally during the staple firing stroke.

Example 22—The stapling assembly of Example 21, wherein the firing driver is configured to retract the first component during the retraction stroke.

Example 23—The stapling assembly of Example 21, wherein the firing driver is configured to retract the second component during the retraction stroke.

Example 24—The stapling assembly of Example 23, wherein the firing driver is configured to operably attach to the second component during the staple firing stroke, and wherein the firing driver is configured to operably detach from the second component during the retraction stroke.

Example 25—The stapling assembly of Example 24, wherein the firing driver comprises at least one lock arm configured to releasably couple the firing driver to the second component.

Example 26—The stapling assembly of Example 23, wherein the first component is unconnected to the firing driver, and wherein the first component is not retracted with the firing driver during the retraction stroke.

Example 27—The stapling assembly of Examples 20, 21, 22, 23, 24, 25, or 26, wherein the first component comprises a protrusion, wherein the second component comprises a cutout engaged with the protrusion to couple the second component with the first component when the sled assembly is in the first configuration, and wherein the cutout is disengaged from the protrusion when the sled assembly is in the second configuration.

Example 28—The stapling assembly of Example 27, wherein the rail of the first component comprises a first rail, wherein the first component further comprises a second rail extending from the base, wherein the second rail comprises a second distal-facing ramp, wherein the second distal-facing ramp is configured to engage the staple drivers to eject the staples from the staple cavities during the staple firing stroke, and wherein the protrusion extends between the first rail and the second rail.

Example 29—The stapling assembly of Examples 18, 19, 20, 21, 22, 23, 24, 25, 26, 27, or 28, wherein the second component comprises plastic overmolded onto the metal of the rail.

Example 30—The stapling assembly of Examples 18, 19, 20, 21, 22, 23, 24, 25, 26, 27, 28, or 29, wherein the rail of the first component comprises a first rail, wherein the first component further comprises a second rail extending from the base, wherein the second rail comprises a second distal-facing ramp, wherein the second distal-facing ramp is configured to engage the staple drivers to eject the staples from the staple cavities during the staple firing stroke, wherein the second rail is comprised of metal, and wherein the first rail is positioned on a first lateral side of the longitudinal slot and the second rail is positioned on a second lateral side of the longitudinal slot.

Example 31—The stapling assembly of Example 30, wherein the first component further comprises a third rail extending from the base, wherein the third rail comprises a third distal-facing ramp, wherein the third distal-facing ramp is configured to engage the staple drivers to eject the staples from the staple cavities during the staple firing stroke, wherein the third rail is comprised of plastic, and wherein the third rail is positioned on a first lateral side of the longitudinal slot.

Example 32—The stapling assembly of Example 31, wherein the first rail is positioned closer to the longitudinal slot than the third rail.

Example 33—The stapling assembly of Examples 18, 19, 20, 21, 22, 23, 24, 25, 26, 27, 28, 29, 30, 31, or 32, wherein the second component comprises a knife.

Example 34—The stapling assembly of Examples 18, 19, 20, 21, 22, 23, 24, 25, 26, 27, 28, 29, 30, 31, 32, or 33, wherein the stapling assembly is replaceable.

Example 35—A stapling system comprising a stapling instrument and a staple cartridge. The stapling instrument comprising a first jaw, a second jaw rotatable relative to the first jaw between an unclamped position and a clamped position, and a firing driver movable distally during a firing stroke and movable proximally during a retraction stroke. The staple cartridge is positioned in the first jaw. The staple cartridge comprises a cartridge body, staple drivers, staples, and a sled assembly. The cartridge body comprises a deck, a longitudinal slot, staple cavities defined in the deck, and a longitudinal recess. The deck includes a proximal end and a distal end. The longitudinal slot extends from the proximal end toward the distal end. The longitudinal recess extends laterally from the longitudinal slot. The staple drivers are movably positioned in the staple cavities. The staples are removably stored in the staple cavities. The sled assembly comprises a ramp portion and a central nose portion. The ramp portion is configured to engage the staple drivers to eject the staples from the staple cavities during the firing stroke. The ramp portion comprises metal. The central nose portion is movable within the longitudinal slot. The central nose portion comprises a plastic portion and a knife. The plastic portion comprises a lateral protrusion. The ramp portion and the central nose portion are moved from a proximal position to a distal position by the firing driver during the firing stroke. Only the central nose portion is retracted from the distal position to the proximal position by the firing driver during the retraction stroke. The lateral protrusion is configured to be supported by the longitudinal recess as the central nose portion moves between the proximal position and the distal position.

Example 36—The stapling system of Example 35, wherein the knife comprises a metal knife blade.

Example 37—A staple cartridge comprising a cartridge body, a staple driver, staples, and a sled assembly. The cartridge body comprises a deck, a longitudinal slot, and a staple cavity defined in the deck. The deck includes a proximal end and a distal end. The longitudinal slot extends from the proximal end toward the distal end. The staple driver is movably positioned in the staple cavity. The staple driver is movable from an unfired position to a fired position. The staples are removably stored in the staple cavities. The sled assembly is movable distally through a firing stroke by a firing driver to move the staple driver from the unfired position to the fired position. The sled assembly comprises a first portion and a second portion. The first portion comprises ramps. The ramps are configured to engage the staple driver and move the staple driver from the unfired position to the fired position to eject the staples from the staple cavities during the firing stroke. The second portion is movable within the longitudinal slot. The second portion comprises a knife. The first portion and the second portion are moved from a proximal position to a distal position by the firing driver during the firing stroke. The first portion and the second portion are moved from the distal position toward the proximal position by the firing driver during a retraction stroke. The first portion and the second portion operably decoupled from one another when the first portion engages the staple driver in the unfired position such that only the second portion is retracted proximally into the proximal position by the firing driver during the retraction stroke.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Patent No. D974,560, entitled STAPLE CARTRIDGE, which issued on Jan. 3, 2023, U.S. Design Patent No. D967,421, entitled STAPLE CARTRIDGE, which issued on Oct. 18, 2022, U.S. Design Patent No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

The surgical instrument systems described herein have been described in connection with the deployment and deformation of staples; however, the present disclosure may not be so limited. The present disclosure envisions that fasteners other than staples can be deployed, such as clamps or tacks, for example. Moreover, the present disclosure envisions utilizing any suitable means for sealing tissue. An end effector in accordance with the present disclosure can comprise electrodes configured to heat and seal the tissue. Also, an end effector in accordance with the present disclosure can apply vibrational energy to seal the tissue.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

It is worthy to note that any reference numbers included in the appended claims are used to reference exemplary embodiments/elements described in the present disclosure. Accordingly, any such reference numbers are not meant to limit the scope of the subject matter recited in the appended claims.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

1-17. (canceled)

18. A stapling assembly, comprising:
a cartridge body, comprising:
a deck including a proximal end and a distal end;
a longitudinal slot extending from said proximal end toward said distal end; and
a plurality of staple cavities defined in said deck;
a plurality of staple drivers movably positioned in said staple cavities;
a plurality of staples removably stored in said staple cavities; and
a sled assembly configurable in a first configuration and a second configuration, wherein said sled assembly is movable distally in said first configuration during a staple firing stroke, wherein said sled assembly is movable proximally in said second configuration during a retraction stroke, and wherein said sled assembly comprises:
a first component, comprising:
a base; and
at least one rail extending from said base, wherein said rail comprises a distal-facing ramp, wherein said distal-facing ramp is configured to engage said staple drivers to eject said staples from said staple cavities during said staple firing stroke, and wherein said rail is comprised of metal; and
a second component movable within said longitudinal slot, wherein said first component is movable relative to said second component.

19. The stapling assembly of claim 18, wherein said second component is comprised of plastic.

20. The stapling assembly of claim 18, wherein said first component and said second component are coupled in said first configuration, and wherein said first component and said second component are decoupled in said second configuration.

21. The stapling assembly of claim 20, further comprising:
a first jaw;
a second jaw rotatable relative to said first jaw between an unclamped position and a clamped position; and
a firing driver movable distally during said staple firing stroke and movable proximally during said retraction stroke, wherein said firing driver moves said sled assembly distally during said staple firing stroke.

22. The stapling assembly of claim 21, wherein said firing driver is configured to retract said first component during said retraction stroke.

23. The stapling assembly of claim 21, wherein said firing driver is configured to retract said second component during said retraction stroke.

24. The stapling assembly of claim 23, wherein said firing driver is configured to operably attach to said second component during said staple firing stroke, and wherein said firing driver is configured to operably detach from said second component during said retraction stroke.

25. The stapling assembly of claim 24, wherein said firing driver comprises at least one lock arm configured to releasably couple said firing driver to said second component.

26. The stapling assembly of claim 23, wherein said first component is unconnected to said firing driver, and wherein said first component is not retracted with said firing driver during said retraction stroke.

27. The stapling assembly of claim 20, wherein said first component comprises a protrusion, wherein said second component comprises a cutout engaged with said protrusion to couple said second component with said first component when said sled assembly is in said first configuration, and wherein said cutout is disengaged from said protrusion when said sled assembly is in said second configuration.

28. The stapling assembly of claim 27, wherein said rail of said first component comprises a first rail, wherein said first component further comprises a second rail extending from said base, wherein said second rail comprises a second distal-facing ramp, wherein said second distal-facing ramp is configured to engage said staple drivers to eject said staples from said staple cavities during said staple firing stroke, and wherein said protrusion extends between said first rail and said second rail.

29. The stapling assembly of claim 18, wherein said second component comprises plastic overmolded onto the metal of said rail.

30. The stapling assembly of claim 18, wherein said rail of said first component comprises a first rail, wherein said first component further comprises a second rail extending from said base, wherein said second rail comprises a second distal-facing ramp, wherein said second distal-facing ramp is configured to engage said staple drivers to eject said staples from said staple cavities during said staple firing stroke, wherein said second rail is comprised of metal, and wherein said first rail is positioned on a first lateral side of said longitudinal slot and said second rail is positioned on a second lateral side of said longitudinal slot.

31. The stapling assembly of claim 30, wherein said first component further comprises a third rail extending from said base, wherein said third rail comprises a third distal-facing ramp, wherein said third distal-facing ramp is configured to engage said staple drivers to eject said staples from said staple cavities during said staple firing stroke, wherein said third rail is comprised of plastic, and wherein said third rail is positioned on a first lateral side of said longitudinal slot.

32. The stapling assembly of claim 31, wherein said first rail is positioned closer to said longitudinal slot than said third rail.

33. The stapling assembly of claim 18, wherein said second component comprises a knife.

34. The stapling assembly of claim 18, wherein said stapling assembly is replaceable.

35. A stapling system, comprising:
a stapling instrument, comprising:
a first jaw;
a second jaw rotatable relative to said first jaw between an unclamped position and a clamped position; and
a firing driver movable distally during a firing stroke and movable proximally during a retraction stroke; and
a staple cartridge positioned in said first jaw, comprising:
a cartridge body, comprising:
a deck including a proximal end and a distal end;
a longitudinal slot extending from said proximal end toward said distal end;
staple cavities defined in said deck; and
a longitudinal recess extending laterally from said longitudinal slot;
staple drivers movably positioned in said staple cavities;
staples removably stored in said staple cavities; and
a sled assembly, comprising:
a ramp portion configured to engage said staple drivers to eject said staples from said staple cavities during said firing stroke, wherein said ramp portion comprises metal; and
a central nose portion movable within said longitudinal slot, wherein said central nose portion comprises:
a plastic portion comprising a lateral protrusion; and
a knife, wherein said ramp portion and said central nose portion are moved from a proximal position to a distal position by said firing driver during said firing stroke, wherein only said central nose portion is retracted from said distal position to said proximal position by said firing driver during said retraction stroke, and wherein said lateral protrusion is configured to be supported by said longitudinal recess as said central nose portion moves between said proximal position and said distal position.

36. The stapling system of claim 35, wherein the knife comprises a metal knife blade.

37. A staple cartridge, comprising:
a cartridge body, comprising:
a deck including a proximal end and a distal end;
a longitudinal slot extending from said proximal end toward said distal end; and
a staple cavity defined in said deck;
a staple driver movably positioned in said staple cavity, wherein said staple driver is movable from an unfired position to a fired position;
staples removably stored in said staple cavities; and
a sled assembly movable distally through a firing stroke by a firing driver to move said staple driver from said unfired position to said fired position, wherein said sled assembly comprises:
a first portion comprising ramps, wherein said ramps are configured to engage said staple driver and move said staple driver from said unfired position to said fired position to eject said staples from said staple cavities during said firing stroke; and
a second portion movable within said longitudinal slot, wherein said second portion comprises a knife, wherein said first portion and said second portion are moved from a proximal position to a distal position by said firing driver during said firing stroke, wherein said first portion and said second portion are moved from said distal position toward said proximal position by said firing driver during a retraction stroke, and wherein said first portion and said second portion operably decoupled from one another when said first portion engages said staple driver in said unfired position such that only said second portion is retracted proximally into said proximal position by said firing driver during said retraction stroke.
